(19) United States
(12) Patent Application Publication
(10) Pub. No.: US 2025/0065048 A1
(43) Pub. Date: Feb. 27, 2025

(54) A sub-assembly of a medicament delivery device

(71) Applicant: SHL Medical AG, Zug (CH)

(72) Inventors: Gary Calderwood, Nacka Strand (SE); Chun Chang, Nacka Strand (SE); Ming-Ting Yin, Taoyuan City (TW); Shang-Yun Chung, Taipei City (TW)

(21) Appl. No.: 18/724,305

(22) PCT Filed: Dec. 23, 2022
(86) PCT No.: PCT/EP2022/087673

§ 371 Date: Jun. 26, 2024

Related U.S. Application Data
(60) Provisional application No. 63/406,408, filed on Sep. 14, 2022.

(30) Foreign Application Priority Data
Jan. 7, 2022 (EP) 21211825.1

(51) Int. Cl. A61M 5/20; A61M 5/31

(52) U.S. Cl. CPC A61M 5/2033; A61M 5/3148

(57) ABSTRACT

The present disclosure provides a sub-assembly of a medicament delivery device, the sub-assembly comprising: a tubular housing, a carrier, a cap, and a carrier retracting assembly. The tubular housing extends along a longitudinal axis between a distal end and a proximal end, and the cap is removably attached to the tubular housing and at least partially enclosing the carrier. The carrier is configured to receive a medicament container of the medicament delivery device. The carrier is movable relative to the tubular housing in the direction of the longitudinal axis from a proximal position where the carrier is at least partially arranged within the tubular housing to a distal position where the carrier is further into the tubular housing by the carrier retracting assembly during a removal of the cap from the tubular housing.

### CROSS-REFERENCE TO RELATED APPLICATIONS

The present application is a U.S. National Phase Application pursuant to 35 U.S.C. § 371 of International Application No. PCT/EP2022/087673 filed Dec. 23, 2022, which claims priority to (i) European Patent Application No. 21211825.1 filed Jan. 7, 2022, and (ii) U.S. Provisional Patent Application No. 63/406,408 filed Sep. 14, 2022. The entire disclosure contents of these applications are herewith incorporated by reference into the present application.

### TECHNICAL FIELD

The present disclosure generally relates to a sub-assembly of a medicament delivery device, and particularly to sub-assembly comprises a carrier retracting assembly.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments, such as a growth hormone.

There are types of medicaments that can be stored for a long time and that are filled in containers, such as cartridges, syringes, ampoules, canisters or the like, containing a ready-to-use medicament in a liquid state. However, other types of medicaments are a mixture of two substances, i.e. a medicament agent (e.g. lyophilized, powdered or concentrated liquid) and a diluent (e.g. water, dextrose solution or saline solution). These types of medicaments cannot be pre-mixed and stored for a long time because the medicament agent is unstable and can be degraded and lose its effect quickly. Hence, a user, e.g. a patient himself/herself, a physician, a nurse, hospital personnel or trained persons, must perform the mixing within a limited period prior to delivery of a dose of medicament to a patient. Further, some medicament agents are subject to chemical changes during mixing. Such sensitive medicament agents require a particular treatment when mixing with a diluent since excessive mixing force will degrade said medicament agents.

To facilitate the mixing, several containers for mixing have been developed to comprise at least two chambers, known as multi-chamber containers. These multi-chambered containers comprise at least a first chamber containing the medicament agent and a second chamber containing the diluent. These chambers are sealed off with stoppers so that the medicament agent is separated from the diluent and does not become degraded. When the medicament agent is to be mixed, shortly before administering, redirecting passages are opened between the chambers, usually by depressing a distal stopper and in turn a divider stopper of the container somewhat. The passages allow the mixing of the medicament agent and the diluent to prepare the medicament for delivery.

To facilitate for the patient to self-administer the medicament with a predetermined dose in an easy, safe and reliable way and also to facilitate the administration of medicaments for hospital personnel in the same facilitated way, several automatic and semi-automatic devices have been developed, incorporating these multiple-chamber solutions, to mix the medicament before delivery.

Even though many of the devices on the market, as well as the ones described above, have their respective advantages, there is still room for improvements, especially improvements regarding safely mixing the medicament and automatically using components and functions that are dedicated to a mixing sequence.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device, the sub-assembly comprising: a tubular housing, a carrier, a cap, and a carrier retracting assembly; the tubular housing extends along a longitudinal axis between a distal end and a proximal end; the cap is removably attached to the tubular housing and at least partially enclosing the carrier; the carrier is configured to receive a medicament container of the medicament delivery device; and the carrier retracting assembly is configured to move the carrier relative to the tubular housing in the direction of the longitudinal axis from a proximal position where the carrier is at least partially arranged within the tubular housing to a distal position where the carrier is further into the tubular housing.

Preferably, according to another embodiment, the carrier retracting assembly comprises a pre-stressed resilient member; the pre-stressed resilient member is arranged between the carrier and the tubular housing and is configured to extend in the direction of the longitudinal axis; the carrier comprises a distally directed stop surface; the sub-assembly comprises a proximally directed stop surface adjacent to the distally directed stop surface of the carrier when the cap is attached to the tubular housing and is offset relative to the distally directed stop surface during the removal of the cap from the tubular housing, so that the carrier is moved from the proximal position to the distal position by the pre-stressed resilient member during the removal of the cap from the tubular housing.

Preferably, according to another embodiment, the tubular housing comprises a fastener; the cap comprises a counter fastener releasably attached to the fastener; and the cap is movable relative to the tubular housing between an attached position where the fastener is fixed to the counter fastener and a detached position where the fastener is not fixed to the counter fastener.

Preferably, according to another embodiment, the fastener and the counter fastener form a bayonet engagement or a screw engagement.

Preferably, according to another embodiment, the proximally directed stop surface is arranged on the cap.

Preferably, according to another embodiment, the sub-assembly comprises an adapter attached to a part of the cap; the proximally directed stop surface is arranged on the adapter; and the adapter is rotatable relative to the carrier around the longitudinal axis.

Alternatively, according to another embodiment, the proximally directed stop surface is arranged on the tubular housing; the cap is rotationally fixed to the carrier by a rotational engagement; and the cap is rotatable around the longitudinal axis relative to the tubular housing during the removal of the cap from the tubular housing.

Preferably, according to another embodiment, the carrier is rotationally fixed to the tubular housing by a rotational engagement.

Preferably, according to another embodiment, the rotational engagement is formed by at least one of a rib-and-rib engagement, a rib-and-recess engagement, and a rib-and-cut-out engagement.

Preferably, according to another embodiment, the tubular housing comprises a distally directed surface; the carrier comprises a proximally directed surface; and the pre-stressed resilient member engages with the distally directed surface of the tubular housing at a proximal end of the pre-stressed member and engages with the proximally directed surface of the carrier at a distal end of the pre-stressed member.

Preferably, according to another embodiment, the tubular housing comprises a proximally directed surface; wherein the carrier comprises a distally directed surface; and the pre-stressed resilient member engages with the distally directed surface of the carrier at a proximal end of the pre-stressed member and engages with the proximally directed surface of the tubular housing at a distal end of the pre-stressed member.

Preferably, according to another embodiment, the pre-stressed resilient member is a compression spring.

Preferably, according to another embodiment, the sub-assembly comprises a rod arranged within the tubular housing; the rod extends along the longitudinal axis between a proximal end and a distal end; the rod is fixed relative to the tubular housing in the direction of the longitudinal axis during the removal of the cap from the tubular housing; and the proximal end of the rod is configured to move a stopper of the medicament container when the carrier is in the distal position.

Preferably, according to another embodiment, the rod is configured to expel the medicament contained within the medicament container.

Preferably, according to another embodiment, the rod is configured to be at least partially surrounded by a spring that is configured to expel the medicament contained within the medicament container.

Preferably, according to another embodiment, the sub-assembly is used in a medicament delivery device comprising a multiple-chamber medicament container arranged within the carrier.

Preferably, according to another embodiment, the medicament delivery device is an injection device, an inhalation device, or a medical sprayer.

Preferably, according to another embodiment, the medicament delivery device is an auto-injector.

Preferably, according to another embodiment, the medicament delivery device is a pen-shaped injector.

Preferably, according to another embodiment, the medicament delivery device comprises a reusable part and a disposable part.

Preferably, according to another embodiment, the disposable part of the medicament delivery device is a cassette where the tubular housing, the cap, the carrier and the carrier retracting assembly are parts of the cassette.

Preferably, according to another embodiment, the auto-injector is configured to provide a subcutaneous injection, an intramuscular injection, or an intravenous injection.

Another aspect of the invention provides a method of operating a medicament delivery device, the method comprising the steps of: providing a medicament delivery device comprising a tubular housing, a carrier receiving a medicament container, a cap, and a carrier retracting assembly; wherein the tubular housing extends along a longitudinal axis between a distal end and a proximal end; and wherein the cap is removably attached to the tubular housing and at least partially encloses the carrier; and removing the cap from the tubular housing, thereby triggering the carrier retracting assembly to retract the carrier further into the tubular housing.

Generally, the medicament delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C—C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a perspective view of a medicament delivery device comprising a sub-assembly of the invention.

FIG. 2 schematically shows the sub-assembly of FIG. 1 when a cap is removed from a tubular housing.

FIGS. 3A-3B schematically show cross-section views of the sub-assembly of FIG. 1 in different configurations.

FIG. 4 schematically shows a perspective view of the tubular housing and a carrier of the sub-assembly of FIG. 1.

FIG. 5 schematically shows a perspective view of the cap of the sub-assembly of FIG. 1.

FIG. 6 schematically shows a perspective view of the cap of FIG. 5 with an adapter.

FIGS. 7A-7C schematically show an exemplified operation sequence of the sub-assembly of FIG. 1.

FIGS. 8A-8B schematically show cross-section views of the medicament delivery device comprising a sub-assembly of FIG. 1.

FIG. 9 schematically shows a perspective view of a drive mechanism of the medicament delivery device of FIG. 9.

FIG. 10 schematically shows a perspective view of components of the drive mechanism of FIG. 9.

FIG. 11 schematically shows a cross-section view of the drive mechanism of FIG. 9.

FIGS. 12A-12C schematically show cross-section views of another example of the sub-assembly of the invention.

### DETAILED DESCRIPTION

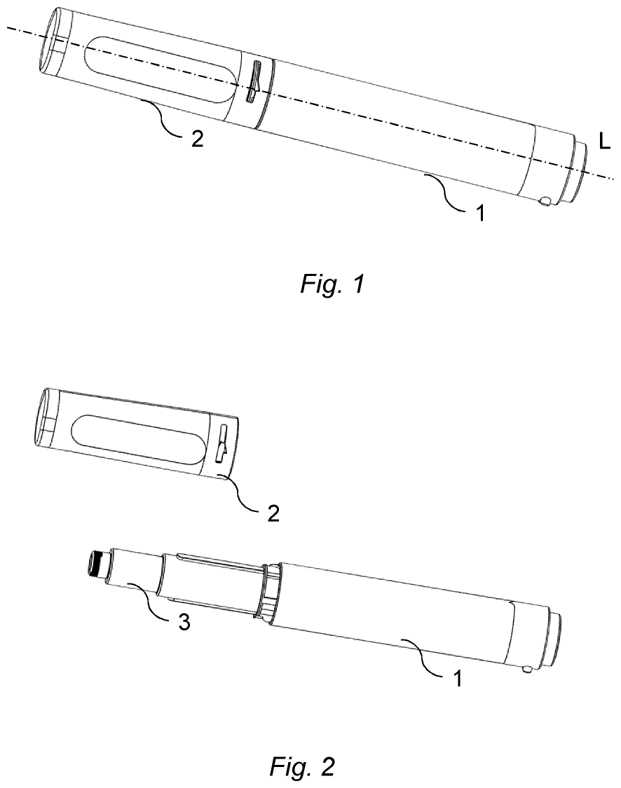

FIGS. 1-11 illustrate a sub-assembly of a medicament delivery device, the sub-assembly comprising: a tubular housing 1, a carrier 3, a cap 2, and a carrier retracting assembly. The tubular housing 1 extends along a longitudinal axis L between a distal end and a proximal end. The cap 2 is removably attached to the tubular housing 1 and at least partially encloses the carrier 2. The carrier 3 is configured to receive a medicament container of the medicament delivery device. The carrier retracting assembly of the sub-assembly is configured to retract the carrier 3 further into the tubular housing 1 during a removal of the cap 2 from the tubular housing 1. The carrier retracting assembly is configured to move the carrier 3 relative to the tubular housing 1 in the direction of the longitudinal axis L from a proximal position where the carrier is at least partially arranged within the tubular housing 1 to a distal position where the carrier 3 is further into the tubular housing 1. The cap 2 is configured to cover a medicament delivery member, e.g. spray nozzle or injection needle, when the cap 2 is attached to the tubular housing 1. The cap 2 can also partially cover the carrier when the carrier is in the proximal position.

The sub-assembly can be used with a medicament delivery device comprising a prefilled syringe or can be used with a medicament delivery device comprising a cartridge that a user needs to attach a separate medicament delivery member to before use of the medicament delivery device.

The carrier retracting assembly is configured to retract the carrier 3 further into the tubular housing 1 during the removal of the cap 2 from the tubular housing 1. For example, the carrier is partially arranged within the tubular housing when the carrier is in the proximal position and is fully arranged within the tubular housing when the carrier is in the distal position. In this example, the cap can be transparent or be arranged with an observation aperture. In this example, if the disclosed sub-assembly is used with a prefilled syringe, the user can observe the medicament delivery member before use when the carrier is in the proximal position, so that the user can check whether the spray nozzle or the injection needle is damaged before use. Once the user removes the cap, the carrier will be retracted further into the tubular housing, namely, the carrier is moved to the distal position, so that the medicament delivery member can be shielded by the tubular housing to avoid any damage to the medicament delivery member and also avoid any injury to the user.

Furthermore, the disclosed sub-assembly can be used with a medicament delivery device that needs to expel any contained air within the medicament container, namely, needs a priming shot. Furthermore, the disclosed sub-assembly can also be used with a medicament container comprising more than one isolated chamber, namely, a multi-chamber medicament container. These chambers are usually isolated from one another by rubber stoppers. The sub-assembly can be used for performing an auto-mixing function of the substances in the different chambers. If the sub-assembly is used with a medicament delivery device that needs a priming shot and/or has an auto-mixing function, the sub-assembly further comprises a rod 6 arranged within the tubular housing 1. The rod is axially immovable relative to the tubular housing 1 when the auto-mixing and/or priming shot takes place. The rod can be configured to move into the medicament container and expel the medicament into the user's body. In this example, the rod will normally be called a plunger rod. Alternatively, the rod 6 can be configured to support the plunger rod or to support a spring that is configured to expel the medicament contained within the medicament container. In other words, the rod itself will not move in the proximal direction relative to the tubular housing to expel the contained medicament. In this example, the rod 6 will normally be called a guide rod 6. The plunger rod and the guide rod will be explained in detail later.

In an example where the disclosed sub-assembly is used for performing an auto-mixing function in a multi-chamber medicament container. The carrier retracting assembly is configured to move the carrier from the proximal position to the distal position during the removal of the cap from the tubular housing. Thereby, the rod of the sub-assembly can act on a stopper of the medicament container, the stopper is arranged at a distal end of the medicament container, when the carrier is in the distal position. Therefore, the substances contained within different chambers can be mixed. Similarly, if the disclosed sub-assembly is used for performing a priming shot, the rod of the sub-assembly acts on the stopper of the medicament container when the carrier is in the distal position, thus the contained air within the medicament container will be expelled.

Therefore, after the user removes the cap, the medicament of the medicament delivery device with the sub-assembly is ready to be used, e.g. fully mixed and/or primed, and no extra operation steps for mixing multiple substances and/or performing a priming shot are needed.

Furthermore, most multi-chamber medicament containers are cartridges, meaning that the user needs to attach a medicament delivery member before use. If the user attaches the medicament delivery member onto the medicament container before completing a mixing operation, then the medicament might leak via the medicament delivery member during the mixing operation, due to increased pressure within the medicament container. The disclosed sub-assembly uses the cap to enclose the carrier so that the user cannot access and attach a medicament delivery member onto the medicament container before mixing is carried out. Once the cap is removed, the mixing operation is also completed, since the carrier is retracted during the cap removal, meaning that the risk of medicament leakage can be mitigated.

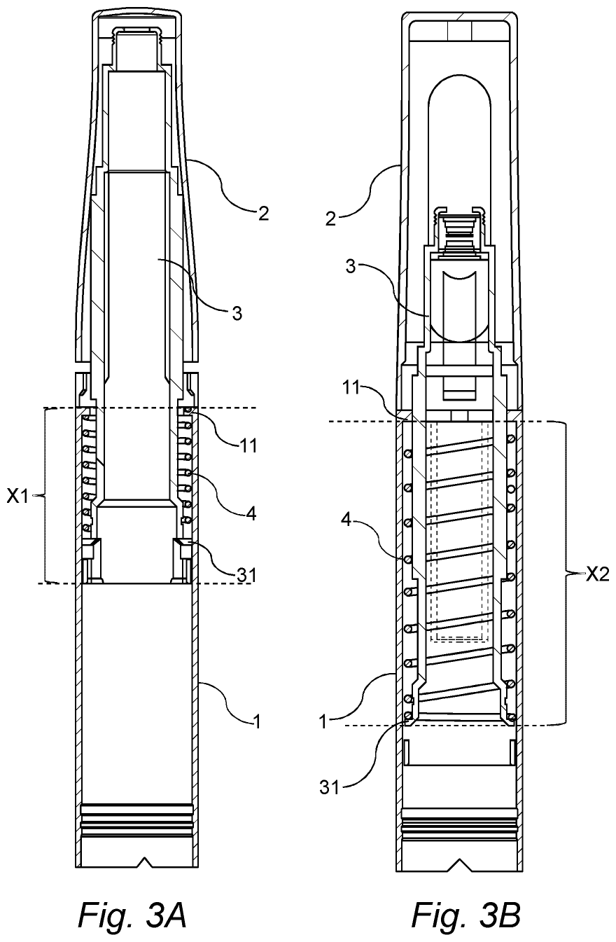

In the example as shown in FIGS. 3A-3B, the carrier retracting assembly comprises a pre-stressed resilient member 4, e.g. a spring or a flexible arm. In the presented example, the pre-stressed resilient member 4 is a coil spring, e.g. a compression spring or a tension spring, the detailed arrangement of which will be explained in detail later. Alternatively, in another example where the spring is a band spring, the carrier retracting assembly may comprise the band spring, a fastener for attaching an end of the band spring to either the carrier 3 or the tubular housing 1, and a pin that the band spring winds around. The pin can be attached to the housing, when the fastener is attached to the carrier. Alternatively, the pin can be attached to the carrier when the fastener is attached to the housing.

Alternatively, the carrier retracting assembly can be formed in any suitable way to retract the carrier further into the housing during cap removal. For example, the carrier retracting assembly can be a gear wheel and racks. The gear wheel and the racks can both be arranged between the cap and the carrier, so that when the cap moves in the proximal direction, the gear wheel moves the carrier, via the rack, in the distal direction to the distal position. Alternatively, the carrier retracting assembly can be a motor gear with a switch configured to be switched on by the cap removal so that the motor gear can move the carrier to the distal position once the switch has been switched on.

In one example where the retracting assembly comprises a pre-stressed resilient member, the cost of the sub-assembly can be reduced in comparison with the example where the retracting assembly comprises a gear wheel and racks or comprises a motor gear and switch.

It should be noted that how far the carrier should be retracted further into the housing (i.e. the distance between the proximal position and the distal position measured in the direction of the longitudinal axis L) is dependent on the design. For example, if the sub-assembly is used in a medicament delivery device that is configured to retract a prefilled syringe further into the housing during cap removal, then the retraction distance of the carrier should be equal or greater than the length of the medicament delivery member. For example, the medicament delivery member is an injection needle with a length 13 mm. The retraction distance of the carrier can then be set as 15 mm for the injection needle with the length 13 mm. Alternatively, if the sub-assembly is used in a medicament delivery device to perform a priming shot, the retraction distance of the carrier is dependent on the volume of a priming shot, which is usually dependent on the contained medicament and the process of medicament filling, e.g. how much gas will be generated or how much air will be sealed within the medicament container. Alternatively, if the sub-assembly is used in a medicament delivery device, the sub-assembly is configured to perform an auto-mixing operation, and the retraction distance of the carrier is dependent on the length needed for squeezing the substance in one or two chambers fully into another chamber. The length is dependent on the types of substances, e.g. liquid or powder, viscosity, volume, along with how many chambers are provided. The retraction distance of the carrier can be fixed by any suitable arrangement. For example, the retraction distance of the carrier can be fixed by arranging a rib and groove between an outer surface of the carrier and an inner surface of the tubular housing. Alternatively, the retraction distance of the carrier can be fixed by the carrier retracting assembly. For example, if the carrier retracting assembly comprises a gear wheel and a rack, the retraction distance of the carrier can be fixed by the length of the rack or the number of teeth on the gear wheel. In another example, the retraction distance of the carrier can be varied by varying the accumulated force of the pre-stressed resilient member.

A detailed description of a preferred embodiment of the disclosed sub-assembly is set forth below.

As shown in FIG. 1, the tubular housing 1 extends along a longitudinal axis L between a distal end and a proximal end. The tubular housing 1 is configured to receive the carrier 3 and is configured to connect to a drive mechanism of the medicament delivery device. The drive mechanism of the medicament delivery device will be explained in detail later.

The carrier 3 is configured to receive a medicament container of the medicament delivery device and is partially arranged within the tubular housing 1 from the proximal end of the tubular housing 1, as shown in FIG. 2. In this example, the carrier 3 is generally cylindrical and comprises a distally directed surface at its proximal inner section for supporting a shoulder section of the medicament container. Alternatively, the carrier 3 can be any suitable shape that can contain a medicament container. For example, most medicament containers are cylindrical, so the carrier can be cylindrical. In another example, a medicament container with two or more chambers has a bump on its outer surface as a bypass, so that when the substance in one chamber is pushed, the substance can flow into another chamber via the bypass. As a result, the carrier is dimensioned to be able to receive the medicament container; or can be an oval tube, so that the medicament container with one or more bumps can be received within the carrier. Alternatively, the carrier can be a ring or a circlip clamped on the outer surface of the medicament container.

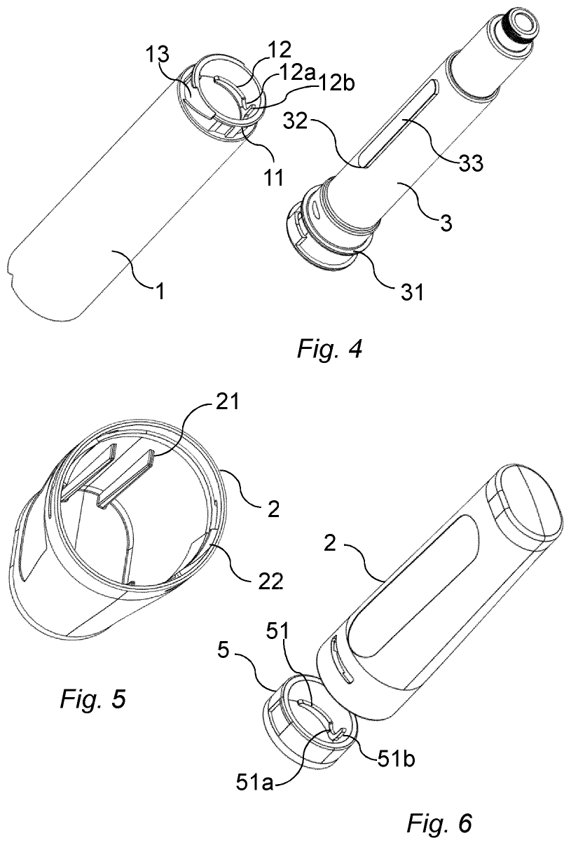

The cap 2 is removably attached to the tubular housing 1 and partially encloses the carrier 3, as shown in FIGS. 1-2. In a preferred example, the cap 2 and the carrier 3 are transparent so that the user can easily observe the medicament container within the carrier 3. Alternatively, the cap comprises an observation window. In the example as shown in FIGS. 1-2, the cap 2 is tubular and has a generally oval cross-section when is observed in the direction along longitudinal axis L, so that the medicament delivery device will not roll when placed on a flat surface. Alternatively, the cap 2 can be any suitable shape depending on the shape of the carrier and/or the shape of the housing and/or the targeted user group. For example, the cap can be tubular, cup shaped or cone shaped. Furthermore, in the example as shown in FIGS. 3A-3B, the cap 2 and the tubular housing 1 are configured to enclose the entire carrier 3. In this example, the tubular housing 1 comprises a fastener 13, as shown in FIG. 4, and the cap 2 comprises a counter fastener 22 releasably attached to the fastener 13. The fastener 13 is arranged on an outer surface of a tubular housing wall, and the counter fastener 22 is arranged on an inner surface of a cap wall. In this example, the cap 2 is movable relative to the tubular housing 1 between an attached position where the fastener 13 is fixed to the counter fastener 22 and a detached position where the fastener 13 is not fixed to the counter fastener 22. The fastener 13 and the counter fastener 22 form a bayonet engagement or a screw engagement. In this example, the cap 2 is configured to be removed from the housing by a rotation around the longitudinal axis L and a subsequent axial movement in the proximal direction relative to the tubular housing 1. Alternatively, in another example, the fastener can be a recess/cut-out and the counter fastener can be a bump positioned within the recess/cut-out. In this example, the cap 2 is configured to be removed from the tubular housing 1 by an axial movement in the proximal direction relative to the tubular housing 1.

The carrier retracting assembly is configured to move the carrier 3 from the proximal position to the distal position during the removal of the cap from the tubular housing 1. In the preferred embodiment, the carrier retracting assembly comprises the pre-stressed resilient member 4 arranged between the carrier 3 and the tubular housing 1, as shown in FIGS. 3A-3B. In this embodiment, the carrier 3 comprises a distally directed stop surface 32 and the sub-assembly comprises a proximally directed stop surface adjacent to the distally directed stop surface 32 of the carrier 3 when the cap 2 is attached to the tubular housing 1 and is offset relative to the distally directed stop surface 32 during the removal of the cap 2 from the tubular housing 1. As mentioned above, the removal of the cap from the tubular housing might comprise a rotational movement and an axial movement of the cap relative to the tubular housing, or only an axial movement of the cap relative to the tubular housing. The carrier retracting assembly can be activated by only the rotational movement, only the axial movement, or both, meaning that the proximally directed stop surface is offset relative to the distally directed stop surface 32 when the cap is rotated relative to the tubular housing around the longitudinal axis, is axially moved relative to the tubular housing or both, depending on the design.

The distally directed stop surface 32 of the carrier 3 can be defined by a distal edge of the carrier, a protrusion 33 on an outer surface of the carrier 3, as shown in FIG. 4, or an edge of a recess/cut-out that is arranged in a sidewall of the carrier.

In one example, the proximally directed stop surface 12 is arranged on the tubular housing 1, as shown in FIG. 4, the cap 2 is rotationally fixed to the carrier 3 through a rotational engagement (which can be formed as the above-mentioned example rotational engagement), as a result, the cap will rotate with the carrier around the longitudinal axis L. For example, a rib 33 is formed on the outer surface of the sidewall of the carrier 3 and a counter rib 21 is formed on the inner surface of the sidewall of the cap 2, as shown in FIGS. 4-5. In this example, the cap 2 is rotatable around the longitudinal axis L relative to the tubular housing 1 during the cap removal from the tubular housing 1. In this example, when the cap 2 rotates, the cap 2 also rotates the carrier 3 via the rotational engagement in between. The carrier 3 is thereby rotated to offset the distally directed stop surface 32 from the proximally directed stop surface 12 of the tubular housing 1 in direction of the longitudinal axis L.

In another example the proximally directed stop surface 51 is arranged on the adapter 5, as shown in FIG. 6, the rotation of the cap 2 results in a rotation of the proximally directed stop surface 51 relative to the distally directed stop surface of the carrier 32. In this example, there is no rotational connection between the cap 2 and the carrier 3, as a result, the cap 2 can rotate relative to the carrier 3 around the longitudinal axis L. For example, the cap can have a diameter that is great enough so that except the proximally directed stop surface, the inner surface of the cap will not contact an outer surface of the carrier. Alternatively, in a preferred example, the carrier 3 is rotationally fixed to the tubular housing 1 through a rotational engagement. The rotational engagement can be formed by a rib-and-rib engagement, a rib-and-recess engagement, or a rib-and-cut-out engagement. For example, the carrier comprises a rib arranged on the outer surface of a sidewall of the carrier and a recess is arranged on an inner surface of the sidewall of the tubular housing. The rib of the carrier is positioned within the recess on the inner surface on the sidewall of the tubular housing, so the carrier cannot be rotated relative to the tubular housing. Alternatively, the rotational engagement can be a match of shapes between the carrier and the tubular housing, for example, the carrier can be oval shape, rectangular shape, or triangle shape when observed in the direction along longitudinal axis L; and the tubular housing can be a corresponding oval shape, rectangular shape, or triangle shape when observed in the direction along the longitudinal axis L.

Furthermore, in the example where the proximally directed stop surface 51 is arranged on the adapter 5, the sub-assembly doesn't need to arrange with the fastener on the tubular housing and the counter fastener on the cap as mentioned above, because the engagement between the distally directed stop surface on the carrier and the proximally directed stop surface on the cap or the adapter can keep the cap attached to the tubular housing.

It should be noted that the proximally directed stop surface as described above is also suitable for being arranged on the cap.

It should be noted that, the proximally directed surface can be arranged on any suitable component that is axially immovable relative to the housing at least before the removal of the cap from the housing. As mentioned above, the proximally directed stop surface can be arranged on the cap, on the tubular housing or on an adapter that attaches to the cap. The proximally directed stop surface can be defined by a ledge extending from an inner surface of a sidewall of the cap, the tubular housing or the adapter; or a recess/cut-out that is arranged in the wall of the cap, the tubular housing or the adapter 5.

The proximally directed stop surface is configured to block the distal movement of the carrier 3 in the direction of the longitudinal axis relative to the tubular housing 1, against a force from the pre-stressed resilient member 4 that moves the carrier 3 in the distal direction relative to the tubular housing 1. A relative rotation around the longitudinal axis L between the proximally directed stop surface and the distally directed stop surface 32 of the carrier 3 is triggered during the removal of the cap 2 from the tubular housing 1.

In an example where the cap 2 is configured to be removed from the tubular housing 1 by a rotation around the longitudinal axis and an axial movement in the proximal direction relative to the tubular housing 1, the proximally directed stop surface can be arranged on the cap, the housing or the adapter 5. In a preferred example, the proximally directed stop surface is offset relative to the distally directed stop surface 32 when the cap 2 is rotated relative to the tubular housing 1 around the longitudinal axis L. In an example where the proximally directed stop surface 51 is arranged on the cap or the adapter, the cap 2 is rotatable relative to the carrier, so that when the cap 2 is rotated relative to the tubular housing 1 around the longitudinal axis L for initiating the cap removal, the rotation of the cap 2 places the proximally directed stop surface 51 offset relative to the distally directed stop surface 32 of the carrier 3 in the direction of the longitudinal axis L.

Furthermore, as shown in FIG. 4 and FIG. 6, the proximally directed stop surface 12 (51) optionally comprises a recess portion 12a (51a) and/or a protrusion portion 12b (51b). The recess portion 12a (51a) opens in the direction of the longitudinal axis L. The protrusion portion 12b (51b) extends in the direction of the longitudinal axis L. The recess portion 12a (51a) is configured to prevent an unintentional relative rotation between the distally directed stop surface 32 of the carrier 3 and the proximally directed stop surface 12 (51). In a preferred example, the distally directed stop surface 32 is convex, and the recess portion 12a (51a) of the proximally directed stop surface 12 (51) is concave to match with the convex surface. Therefore, a potential unintentional relative rotation between the distally directed stop surface 32 of the carrier 3 and the proximally directed stop surface 12 (51), for example when the sub-assembly is shaken, can be prevented. The protrusion portion 12b; 51b of the proximally directed stop surface 12 (51) is configured to limit the rotational direction of the relative rotation between the distally directed stop surface 32 of the carrier 3 and the proximally directed stop surface 12 (51). Thus, the distal movement of the carrier to the distal position can only be triggered when the proximally directed stop surface 12 (51) is rotated in one direction relative to the distally directed stop surface 32 of the carrier 3; or the distally directed stop surface 32 of the carrier 3 is rotated in one direction relative to the proximally directed stop surface 12 (51).

Furthermore, in one example where the proximally directed stop surface 51 is arranged on the adapter 5, which is the component that attaches to the cap 2, the cap can be arranged to be removed from the housing by either a rotational movement and an axial movement or only an axial movement. For example, the adapter (e.g. a ring, a circlip, or a cylinder), is arranged within the cap. In one example, the adapter comprises a helical track or a helical cam surface; and the cap comprises a protrusion extending from an inner surface of a sidewall of the cap. The protrusion of the cap is configured to engage with the helical track or the helical cam surface. Therefore, during the cap removal from the tubular housing, the protrusion of the cap will move along the helical track or helical cam surface, thereby the axial movement of the cap rotates the adapter. The rotation of the adapter positions the proximally directed stop surface at an offset relative to the distally directed stop surface of the carrier. Alternatively, the cap can be arranged with the helical track or the helical cam surface, and the adapter can be arranged with the protrusion, dependent on the design choice.

In another example, the adapter 5 is rotated by the rotation of the cap 2 relative to the tubular housing. In this example, the adapter can be simply fixed to the cap or can be arranged to rotate with a different rate or rotational angle relative to the cap than relative to the tubular housing. In one example where the adapter is fixed to the cap, this example can provide manufacturing efficiency since the cap can be standardized for most medicament delivery devices. In one example where the adapter is arranged to rotate with a different rate or rotational angle relative to the cap than relative to the tubular housing, this example can provide greater design flexibility. For example, the adapter is arranged with a circumferential groove and the cap comprises a protrusion positioned within the groove so that the cap rotates relative to the adapter first, then contacts an edge of the groove, then the cap and the adapter can rotate together. In this example, if the user starts to twist the cap but then immediately decides not to carry out a medicament delivery operation, the retraction of the carrier will not be triggered. Alternatively, a similar effect can be provided by the design of the length of the proximally directed stop surface in a circumferential direction.

In one example, the carrier retracting assembly comprises the pre-stressed resilient member 4 arranged between the carrier and the tubular housing. In one example, the pre-stressed resilient member is a compression spring 4, as shown in FIGS. 3A-3B, and the compression spring 4 extends along the longitudinal axis between a proximal end and a distal end. The tubular housing 1 comprises a distally directed surface 11 and the carrier 3 comprises a proximally directed surface 31, as shown in FIGS. 3A-3B. The pre-stressed resilient member 4 engages with the distally directed surface 11 of the tubular housing 1 at its proximal end and engages with the proximally directed surface 31 of the carrier 3 at its distal end. Therefore, when the carrier 3 is in the proximal position, as shown in FIG. 3A, the distally directed stop surface 32 of the carrier 3 is adjacent to the proximally directed stop surface 12; 51, the pre-stressed resilient member 4 is stressed, and a portion X1 of the carrier 3 is arranged within the tubular housing 1, as shown in FIG. 3A. When the distally directed stop surface 32 of the carrier 3 is offset relative to the proximally directed stop surface (e.g. on the housing, on the cap or on the adapter), the compression spring 4 relaxes; thus, the compression spring 4 pushes the carrier 3 to the distal position of the carrier, as shown in FIG. 3B. The carrier 3 is further moved into the tubular housing 1, and a portion X2 of the carrier 3 is arranged within the tubular housing 1. The portion X2 is longer than the portion X1 when measured in the direction of the longitudinal axis L, and the retraction distance of the carrier 3 is defined by the difference between the X1 and X2.

Alternatively, the pre-stressed resilient member is a flexible arm arranged between the tubular housing and the carrier in the direction of the longitudinal axis L. Alternatively, the pre-stressed resilient member 4 is a tension spring.

In one example where the pre-stressed resilient member is a tension spring, the tension spring extends along the longitudinal axis L between a proximal end and a distal end. The tubular housing comprises a proximally directed surface and the carrier comprises a distally directed surface. The pre-stressed resilient member engages with the distally directed surface of the carrier at its proximal end and engages with the proximally directed surface of the tubular housing at its distal end. Therefore, when the distally directed stop surface of the carrier is offset relative to the proximally directed stop surface (e.g. on the housing, on the cap or on the adapter), the tension spring can pull the carrier further into the tubular housing. The tension spring can be a coil tension spring or a stressed band spring, for example.

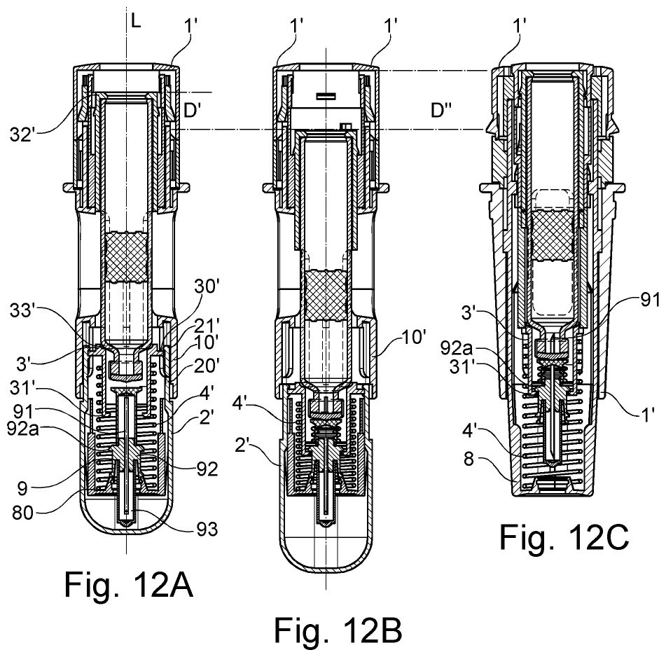

Another example of the sub-assembly of the invention as shown in FIGS. 12A-12C. In this example, the tubular housing 1′ comprises an inner wall 10′ facing towards the longitudinal axis L of the tubular housing 1′. In a preferred example, the inner wall 10′ is located closer to the proximal end of the tubular housing 1′ than the distal end of the tubular housing 1′. The cap 2′ is removable attached to the tubular housing 1′. In this example, the carrier 3′ is axially and bidirectionally movable relative to the tubular housing 1′ in the direction of the longitudinal axis L. As mentioned above, the carrier 3′ is configured to receive a medicament container. In a preferred example, the carrier 3′ is configured to partially receive the medicament container. In this example, the cap 2′ comprises a flexible wall 20′. A protrusion 21′ extends from the flexible wall 20′ of the cap 2′ towards the longitudinal axis L. The flexible wall 20′ is positioned between the inner wall of the tubular housing 1′ and the carrier 3′ when the cap 2′ is attached to the tubular housing 1′. The protrusion 31′ of the flexible wall 20′ of the cap 2′ is lined up with the inner wall 10′ of the tubular housing 1′ in the direction transverse to the longitudinal axis L.

Thus, flexible wall 20′ is at partially adjacent to the inner wall 10′ of the tubular housing 1′ when the cap 2′ is attached to the tubular housing 1′. The cap 2′ comprises a protrusion extending from the flexible wall towards the longitudinal axis L. The protrusion 31′ defines a proximally directed surface. The carrier 3′ comprises a distally directed surface. In one example, the carrier 3′ comprises a protrusion 30′ extending towards the flexible wall of the cap 2′ and the distally directed surface is defined by the protrusion of the carrier 3′. Alternatively, a recess/cut-out is arranged in the carrier and open towards the flexible wall of the cap. In this example, the distally directed surface is defined by an edge of the recess/cut-out.

As shown in FIGS. 12A-12B, the proximally directed surface of the cap is adjacent to the distally directed surface of the carrier 3′ when the cap 2′ is attached to the tubular housing 1′. When the cap 2′ is moved in the proximal direction relative to the tubular housing 1′, the proximally directed surface of the cap 2′ engages with the distally directed surface of the carrier 3′. The interface formed between the proximally directed surface of the cap 2′ and the distally directed surface of the carrier 3′ is bevelled relative to the longitudinal axis L.

In this example, the carrier retracting assembly comprises a resilient member 4′, e.g., a compression spring 4′. The compression spring 4′ is extending from a proximally directed surface of the carrier 3′ to a distally directed surface of the tubular housing 1′, and the compression spring 4′ is compressible between the proximally directed surface of the carrier 3′ to the distally directed surface of the tubular housing 1′. In this example, the cap 2′ is configured to be removed from the tubular housing 1′ by being moved in the proximal direction relative to the tubular housing 1′. In a preferred example, the cap 2′ is configured to be manually pulled by a user in the proximal direction relative to the tubular housing 1′. When the flexible wall 20′ of the cap 2′ is adjacent to the inner wall 10′ of the tubular housing 1′, the inner wall 10′ of the tubular housing 1′ blocks the flexible wall of the cap 2′ from flexing radially outwards relative to the longitudinal axis L, as shown in FIG. 12A. As the proximally directed surface of the cap 2′ engages with the distally directed surface of the carrier 3′, the cap 2′ and the carrier 3′ are moved in the proximal direction relative to the tubular housing 1′ together. The proximal movement of the carrier 3′ causes the compression spring 4′ to be compressed, as shown in FIG. 12B. When the cap 2′ is moved in the proximal direction relative to the tubular housing 1′ in a first predetermined distance D′, the protrusion of the flexible wall 20′ of the cap 2′ is no longer lined up with the inner wall 10′ of the tubular housing 1′, the flexible wall of the cap 2′ flexes radially outwards relative to the longitudinal axis L. Thus, the proximally directed surface of the cap 2′ disengages from the distally directed surface of the carrier 3′, therefore, the cap 2′ is detached from the tubular housing 1′. Once the cap 2′ is detached from the tubular housing 1′, the compressed compression spring 4′ biases the carrier 3′ relative to the tubular housing 1′in the direction of the longitudinal axis from the proximal position to the distal position, as shown in FIG. 12C. As the carrier 3′ is moved from the proximal position to the distal position with a distance D″, a priming action and/or a mixing action can be provided.

It should be noted that instead of arranging the compression spring 4′ between the carrier 3′ and the housing 1′, the compression spring 4′ can be arranged between the carrier 3′ and other component. For example, the sub-assembly comprises a medicament delivery member guard 8. The medicament delivery member guard 8 is telescopic relative to the proximal end of the tubular housing 1′. The medicament delivery member guard 8 is configured to surround a medicament delivery member of the medicament delivery device, e.g., an injection needle, before and after a medicament delivery operation, so that the medicament delivery member can be prevented from being contaminated and the user can be prevented from any injury by the medicament delivery member. In this example, instead of the housing 1′, the carrier retracting assembly comprises a compression spring 4′. The compression spring 4′ is extending from a proximally directed surface of the carrier 3′ to a distally directed surface 80 of the medicament delivery member guard 8, and the compression spring 4′ is compressible between the proximally directed surface of the carrier 3′ to the distally directed surface 80 of the medicament delivery member guard 8. In this example, the compression spring 4′ can provide two functions, both for retracting the carrier 3′ further into the tubular housing 1′ and moves the medicament delivery member guard 8 out of the tubular housing 1′, so that the medicament delivery member guard 8 can surround the medicament delivery member.

In a preferred example, the flexible wall 20′ of the cap 2′ is a flexible arm.

Furthermore, instead of pulling the cap relative to the tubular housing, the cap can be rotated around the longitudinal axis relative to the tubular housing and moved in the proximal direction of the tubular housing. For example, the cap is attached to the tubular housing with a thread engagement, or a helical cam interface formed between the cap and the tubular housing.

In another example, when the medicament container is a cartridge, the sub-assembly comprises a medicament delivery member assembly 9. As shown in FIGS. 12A-12C. The medicament delivery member assembly 9 comprises a medicament delivery member 91, a hub 92 fixed to the medicament delivery member 91, and a sheath 93 sealing the medicament delivery member. The hub 92 comprises a fastener 92a and the carrier 3′ comprises a counter fastener 31′ configured to be attached to the fastener of the hub 92. The hub 92 is movable relative to the tubular housing 1′ in the direction of the longitudinal axis L. In a preferred example, the medicament delivery member assembly 9 is partially surrounded by the compression spring 4′. The medicament delivery member 91 is lined up with the carrier 3′ in the direction of the longitudinal axis L. When the cap 2′ is attached to the housing 1′, the medicament delivery member 91 is spaced part from the carrier 3′ and the proximally arranged to the carrier 3′. In one example, the medicament delivery member assembly 9 is placed on a distally directed surface of the housing 1′. Alternatively, the medicament delivery member assembly is placed on a distally directed surface of the medicament delivery member guard 8 when the cap 2′ is attached to the housing 1′. Therefore, when the cap 2′ is moved in the proximal direction relative to the housing 1′, the cap 2′ also moves the carrier 3′ in the proximal direction relative to the housing 1′ as mentioned above. The carrier 3′ thus is moved towards the medicament delivery member assembly, as shown in FIGS. 12A-12B. Once the carrier 3′ is moved with a second predetermined distance D′, the fastener 92a of the hub is attached to the counter fastener 31′ of the carrier 3′. The engagement formed between the fastener and the counter fastener can be a snap-fit engagement, as shown in FIGS. 12A-12C, or a press-fit engagement. When the carrier 3′ is attached to the hub of the medicament delivery member assembly 9, the medicament delivery member 91 is in fluid communication with the medicament container within the carrier 3′, as shown in FIG. 12C.

In a preferred example, the medicament delivery member 91 is an injection needle.

It should be noted that the first predetermined distance can be the same or different to the second predetermined distance dependent on the design.

Furthermore, in a preferred example, the carrier 3′ comprises a proximally directed support surface 32′ configured to be adjacent to the distal end of the medicament container. Additionally, the carrier 3′ optionally comprises a distally directed support surface 33′ configured to be adjacent to a proximal shoulder of the medicament container.

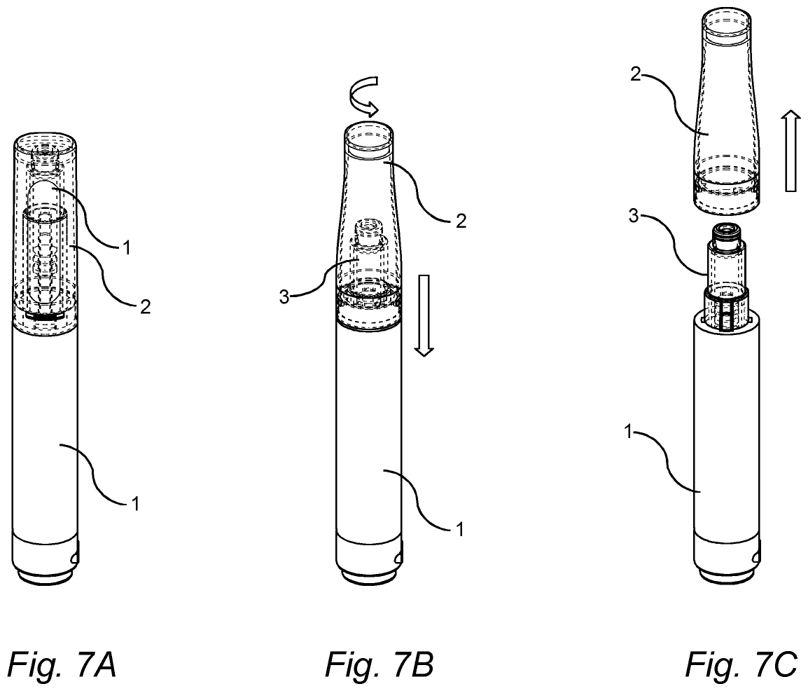

FIGS. 7A-7C illustrate an exemplified operation sequence of the sub-assembly. In this example, the cap 2 is configured to be removed from the tubular housing 1 by rotation around the longitudinal axis L relative to the tubular housing 1 and axial movement in the proximal direction relative to the tubular housing 1. FIG. 7A illustrates the carrier 3 in the proximal position. The rotation of the cap 2 around the longitudinal axis L relative to the tubular housing 1 turns into a relative rotation between the distally directed stop surface 32 of the carrier 3 and the proximally directed stop surface 12; 51. Therefore, the pre-stressed resilient member 4 moves the carrier 3 to the distal position, as shown in FIG. 7B.

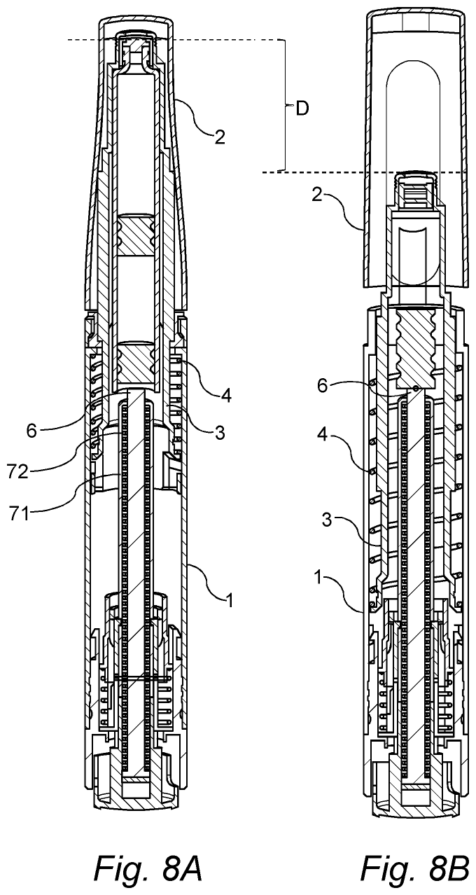

In one example where the sub-assembly is capable of performing an auto-mixing or priming, the sub-assembly comprises the rod 6. The rod 6 is arranged within the tubular housing 1. The rod 6 extends along the longitudinal axis L between a proximal end and a distal end. The rod 6 is fixed relative to the tubular housing 1 in the direction of the longitudinal axis during the removal of the cap from the housing 1. The proximal end of the rod 6 is configured to move a stopper of the medicament container when the carrier 3 is in the distal position, as shown in FIG. 8B. Furthermore, the proximal end of the rod 6 can engage with the stopper of the medicament container when the carrier 3 is in the proximal position, as shown in FIG. 8A, so that a shock usually generated when the rod 6 hits the stopper can be prevented, because the shock might damage the medicament container. On the other hand, the proximal end of the rod can be spaced apart from the stopper when the carrier is in the proximal position, so that if the medicament delivery device with the disclosed sub-assembly is shipped by air freight, even if changes of the atmospheric pressure cause the stopper to move, the stopper will not be blocked by the rod (which could result in unintentional medicament expulsion or mixing).

In the example as shown in FIGS. 8A-8B, the rod can be a guide rod 6 for the medicament delivery device comprising the disclosed sub-assembly, namely, the rod 6 is configured to be surrounded by a spring 72 that is configured to expel the medicament contained within the medicament container. In this example, the guide rod 6 protrudes from a proximal end of the plunger rod 71 of the medicament delivery device, as shown in FIGS. 8A-8B, before a medicament delivery operation starts. Alternatively, the rod is a plunger rod. The rod can be a plunger rod for a medicament delivery device comprising the disclosed sub-assembly, namely, the rod is configured to expel the medicament contained within the medicament container.

In one example where the rod is the guide rod, this example is suitable for being used with a medicament delivery device where the plunger rod is always subjected to a force for expelling the contained medicament, such as a linear spring force. This is because when the plunger rod is pushed by the stopper of the medicament container during the retraction of the carrier, the pushing force might cause the plunger rod to disengage with a holding member (this will be explained in detail later) so that an unintentional trigger of the medicament delivery operation might occur if the plunger rod is always subjected to a force for expelling the contained medicament. In one example where the rod is the plunge rod, this example is suitable for being used with a medicament delivery device that the plunger rod is not always subjected to a force for expelling the contained medicament, such as a motor-driven power source or a gas propellant power source. Because those power sources usually will not output a force to the plunger rod unless the medicament delivery device is triggered.

In the example as shown in FIGS. 8A-8B, the rod 6 is elongated rod-shaped component. Alternatively, the rod 6 (the plunger rod or guide rod) can be formed in any suitable shape, dependent on the design, for example dependent on the selection of the power source of the medicament delivery device and/or dependent on the volume of the contained medicament.

The disclosed sub-assembly can be used with a medicament delivery device comprising a drive mechanism. The drive mechanism of the medicament delivery device comprises a power source, a force transferring member and a trigger assembly. The power source is configured to provide a force for expelling the contained medicament of the medicament delivery device. The force transferring member is configured to transfer the force from the power source to the medicament container. The trigger assembly is configured to either releasably hold the force transferring member or control the output force from the power source.

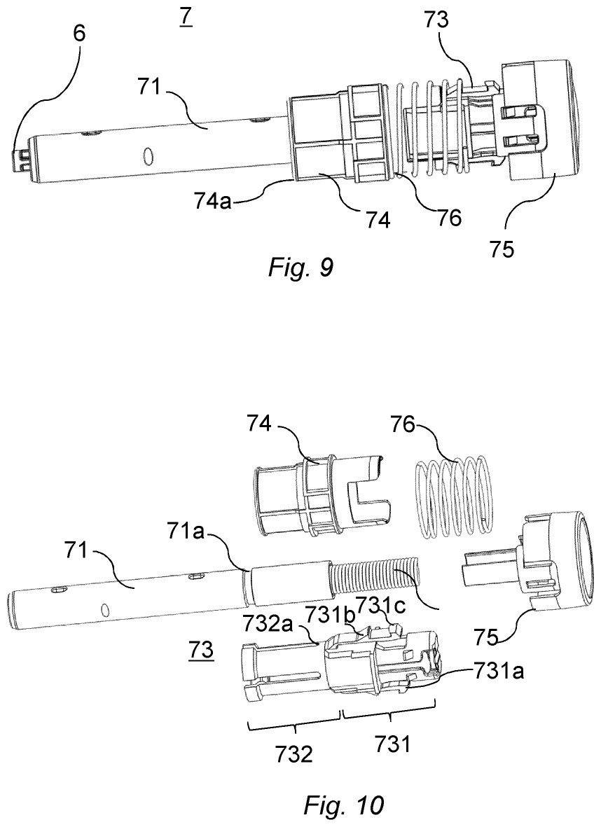

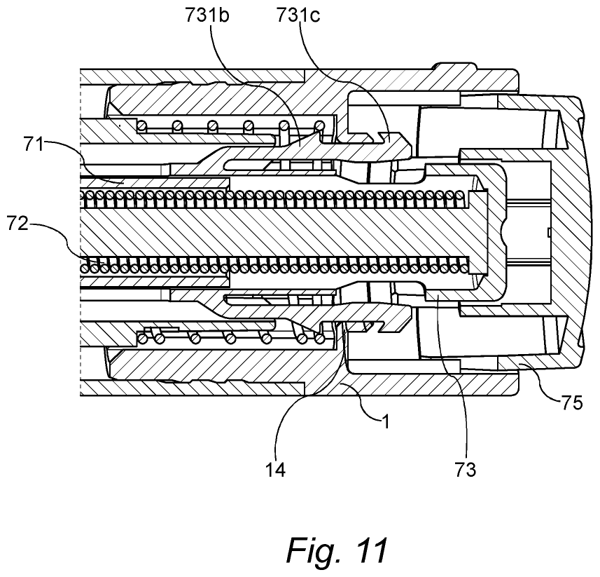

In the preferred example as shown in FIGS. 8A-11, the rod 6 of the sub-assembly is also the guide rod 6 for the drive mechanism. The plunger rod 71 mentioned below is not a part of the sub-assembly as mentioned above. In the preferred example as shown in FIGS. 9-10, the drive mechanism 7 comprises the plunger rod 71 (the plunger rod is the force transferring member in this example), a plunger rod spring 72 (the plunger rod spring is the power source in this example), an actuator 73, an actuator sleeve 74, and a button 75. In this example, the trigger assembly is formed by the actuator 73, the actuator sleeve 74, and the button 75. The button 75 is arranged at the distal end of the tubular housing 1, as shown in FIG. 11, and is axially movable relative to the tubular housing 1 between an initial position and a triggered position. The plunger rod 71 is configured to be biased by the plunger rod spring 72, thereby expelling the contained medicament in the medicament container when the button 75 is in the triggered position. The actuator 73 is configured to releasably engage with the plunger rod 71. The actuator sleeve 74 is configured to release the engagement between the actuator 73 and the plunger rod 71 when the button 75 is in the triggered position. The plunger rod 71 comprises a distally directed support surface and a proximally directed holding surface 71a. The actuator 73 comprises a body 731, 732 and a flexible arm 732a extending from the body 731, 732. The body 731, 732 comprises a proximally directed support surface. The flexible arm 732a comprises a distally directed holding surface adjacent to the proximally directed holding surface 71a of the plunger rod 71. The plunger rod spring 72 is adjacent to the distally directed support surface of the plunger rod 71 at its proximal end and is adjacent to the proximally directed support surface of the actuator 73 at its distal end. The actuator sleeve 74 comprises a sleeve body aligned with the flexible arm 732a of the actuator 73 in a direction transverse to the longitudinal axis L. The drive mechanism 7 optionally comprises a safety mechanism formed by a biasing member 76 arranged between the actuator 73 and the actuator sleeve 74 in the direction of the longitudinal axis L, as shown in FIG. 9, and an enlarged portion on an outer surface of the flexible arm 732a of the actuator 73. Therefore, the flexible arm 732a can only be offset relative to the sleeve body of the actuator sleeve by axially moving in the proximal direction relative to the actuator sleeve 74; also, the biasing member 76 is configured to prevent unintentional proximal movement of the flexible arm 732a of the actuator 73 relative to the actuator sleeve 74.

The button 75 is configured to move the flexible arm 732a of the actuator 73 axially offset relative to the sleeve body of the actuator sleeve 74 when the button 75 is moved from the initial position to the triggered position. In one example, the button 75 is configured to engage and move the body 731, 732 of the actuator 73 out from the body sleeve of the actuator sleeve 74. Optionally, in this example, the body 731, 732 is formed by a proximal body 732 and a distal body 731. The flexible arm 732a extends from the proximal body 731. The distal body 731 comprises a resilient arm 731a extending from the distal body 731. The resilient arm 731a comprises a protrusion 731b extending in the direction transverse to the longitudinal axis and a hook 73 1c comprising a proximally directed hook surface, as shown in FIG. 10. In this example, a distal end of the carrier 3 is configured to engage a proximal end 74a of the actuator sleeve 74 and move the actuator sleeve in the distal direction relative to the tubular housing 1 when the carrier 3 is retracted further into the tubular housing 1. A distal portion of the sleeve body of the actuator sleeve 74 is configured to compress the resilient arm 731a in the radial direction relative to the longitudinal axis L when the carrier 3 moves the actuator sleeve 74 in the distal direction relative to the tubular housing 1. As shown in FIG. 11, the hook 731c will be blocked by a distal wall 14 of the tubular housing 1 if the button 75 presses the actuator 73 in the proximal direction relative to the tubular housing 1 before the resilient arm 731a is compressed in the radial direction relative to the longitudinal axis L. The arrangement as defined in this example provides an optional safety mechanism so that the button 75 can only be pressed to trigger the medicament delivery operation when the carrier is further retracted into the tubular housing 1.

Alternatively, in another example, the button is configured to engage and move the plunger rod in the proximal direction of the tubular housing so that the plunger rod moves the body of the actuator out from the body sleeve of the actuator sleeve through the engagement between the plunger rod and the actuator. In a preferred example, the actuator is configured to surround the plunger rod. The plunger rod comprises an elongated body configured to releasably block the flexible arm of the actuator from flexing radially inward relative to the body of the actuator so that the actuator is engaged with the actuator sleeve and prevented from being moved in the distal direction of the tubular housing until the elongated body of the plunger rod moves out from the body of the actuator.

In another example, the medicament delivery device with the drive mechanism as mentioned above comprises an indication mechanism. In this example, the plunger rod spring 72 is arranged between the plunger rod 71 and the actuator 73, as shown in FIG. 11. In this example, the flexible arm 732a of the actuator 73 comprises an enlarged proximal end. When the plunger rod 71 is surrounded by the flexible arm 732a, the flexible arm 732a is blocked by the plunger rod 71, and thus, cannot flex radially inward relative to the actuator sleeve 74. In this example, the actuator sleeve 74 is configured to engage with the enlarged proximal end of the flexible arm 732a of the actuator 73, when the flexible arm 732a is supported by the plunger rod 71. In this example, the plunger rod spring 72 is configured to axially move the actuator 73 in the distal direction of the tubular housing 1 so that when the plunger rod 71 no longer supports the flexible arm 732a of the actuator 73, the flexible arm 732a flexes radially inward relative to the actuator sleeve 74. Therefore, the enlarged portion of the flexible arm 732a of the actuator 73 will not be blocked by the actuator sleeve 74. An audible indication is generated when the actuator hits an inner surface of the tubular housing 1. The audible indication can be a start indication or an end indication dependent on the design, for example, by adjusting the length of the body of the actuator. For example, the actuator 73 is configured to provide an audible indication for indicating the end of the medicament delivery operation. In this example, the flexible arm 732a of the actuator has a length measured in the direction of the longitudinal axis L that is about a moving distance of the plunger rod for emptying the contained medicament in the medicament container in the direction of the longitudinal axis L. Therefore, when the medicament container is empty, the plunger rod can fully move out from being surrounded by the flexible arm 732a of the actuator 73, and the flexible arm 732a flexes radially inward relative to the actuator sleeve 74. As a result, the actuator 73 can be biased by the plunger rod spring 72 in the distal direction relative to the tubular housing 1. Once a portion of the actuator hits a portion of the tubular housing, for example, a distal end of the protrusion 731b of the actuator 73 hits a proximal end of the distal wall 14 of the tubular housing 1, the end indication is generated, e.g. an end click.

It should be noted that instead of the above-mentioned example, the drive mechanism of the medicament delivery device can be formed in any other suitable way. For example, instead of the plunger rod spring, the power source can be other types of springs, e.g. a torsion spring or a band spring. Alternatively, the power source can be a motor or a gas canister. Similarly, instead of being arranged within the tubular housing 1 as in the above-mentioned examples, the drive mechanism can be packed in an independent power pack housing that connects to the tubular housing. In the example as shown in FIGS. 8A-8B, the tubular housing is cylindrical. Alternatively, it should be noted that the tubular housing I can be any suitable shape depending, for example, on the size of the drive mechanism, and the number of components of the drive mechanism. For example, the tubular housing can be rectangular or triangular when observed along the longitudinal axis L.

The inventive concept has mainly been described above with reference to a few examples. However, as is readily appreciated by a person skilled in the art, other embodiments than the ones disclosed above are equally possible within the scope of the inventive concept, as defined by the appended claims.

Some other aspects of the invention are defined by the following clauses.
1. A sub-assembly of a medicament delivery device, the sub-assembly comprising: a tubular housing (1; 1′), a carrier (3; 3′), a cap (2; 2′), and a carrier retracting assembly (4; 4′);
   wherein the tubular housing (1; 1′) extends along a longitudinal axis (L) between a distal end and a proximal end;
   wherein the cap (2; 2′) is removably attached to the tubular housing (1; 1′) and at least partially enclosing the carrier (3; 3′);
   wherein the carrier (3; 3′) is configured to receive a medicament container of the medicament delivery device; and
   wherein the carrier retracting assembly is configured to move the carrier (3; 3′) relative to the tubular housing in the direction of the longitudinal axis from a proximal position where the carrier is at least partially arranged within the tubular housing (1; 1′) to a distal position where the carrier is further into the tubular housing.
2. The sub-assembly according to clause 1, wherein the carrier (3) retracting assembly comprises a resilient member (4; 4′).
3. The sub-assembly according to clause 2, wherein the pre-stressed resilient member (4) is arranged between the carrier (3) and the tubular housing (1) and is configured to extend in the direction of the longitudinal axis (L); wherein the carrier (3) comprises a distally directed stop surface (32); wherein the sub-assembly comprises a proximally directed stop surface (12; 51) adjacent to the distally directed stop surface (32) of the carrier (3) when the cap (2) is attached to the tubular housing (1) and is offset relative to the distally directed stop surface (32) during the removal of the cap (2) from the tubular housing (1).
4. The sub-assembly according to any of the preceding clauses, wherein the tubular housing comprises a fastener; wherein the cap comprises a counter fastener releasably attached to the fastener; and wherein the cap is movable relative to the tubular housing between an attached position where the fastener is fixed to the counter fastener and a detached position where the fastener is not fixed to the counter fastener.
5. The sub-assembly according to clause 4, wherein the fastener and the counter fastener form a bayonet engagement or a screw engagement.
6. The sub-assembly according to any one of clauses 2-5, wherein the proximally directed stop surface is arranged on the cap.
7. The sub-assembly according to any one of clauses 2-5, wherein the sub-assembly comprises an adapter attached to a part of the cap; wherein the proximally directed stop surface is arranged on the adapter; and wherein the adapter is rotatable relative to the carrier around the longitudinal axis.
8. The sub-assembly according to any one of clauses 2-5, wherein the proximally directed stop surface is arranged on the tubular housing; wherein the cap is rotationally fixed to the carrier by a rotational engagement; and wherein the cap is rotatable around the longitudinal axis relative to the tubular housing during the removal of the cap from the tubular housing.
9. The sub-assembly according to any one of clauses 1-7, wherein the carrier is rotationally fixed to the tubular housing by a rotational engagement.
10. The sub-assembly according to clause 8 or 9, wherein the rotational engagement is formed by at least one of a rib-and-rib engagement, a rib-and-recess engagement, and a rib-and-cut-out engagement.
11. The sub-assembly according to any one of clauses 3-10; wherein the tubular housing comprises a distally directed surface; wherein the carrier comprises a proximally directed surface; and wherein the pre-stressed resilient member engages with the distally directed surface of the tubular housing at a proximal end of the pre-stressed member and engages with the proximally directed surface of the carrier at a distal end of the pre-stressed member.
12. The sub-assembly according to any one of clauses 2-10, wherein the resilient member is a compression spring.
13. The sub-assembly according to any one of the preceding clauses, wherein the sub-assembly comprises a rod arranged within the tubular housing; wherein the rod extends along the longitudinal axis between a proximal end and a distal end; wherein the rod is fixed relative to the tubular housing in the direction of the longitudinal axis during the removal of the cap from the tubular housing; and wherein the proximal end of the rod is configured to move a stopper of the medicament container when the carrier is in the distal position.
14. The sub-assembly according to clause 14 or 15, wherein the rod is configured to be at least partially surrounded by a spring that is configured to expel the medicament contained within the medicament container.
15. The sub-assembly according to clause 1 or 2, wherein the cap is configured to compress the resilient member during cap removal.
16. The sub-assembly according to any of the preceding clauses, comprising a medicament delivery member guard being telescopic relative to the tubular housing.
17. The sub-assembly according to clauses 16 when dependent on clauses 2 or a combination of clauses 15 and clauses 2, wherein the resilient member is configured to bias the medicament delivery member guard in the proximal direction relative to the tubular housing.
18. A method of operating a medicament delivery device, the method comprising the steps of:
   providing a medicament delivery device comprising a tubular housing (1; 1′), a carrier (3; 3′) receiving a medicament container, a cap (2; 2′), and a carrier retracting assembly (4; 4′); wherein the tubular housing (1; 1′) extends along a longitudinal axis (L) between a distal end and a proximal end; and wherein the cap (2; 2′) is removably attached to the tubular housing (1; 1′) and at least partially encloses the carrier (3; 3′); and
   removing the cap from the tubular housing, thereby triggering the carrier retracting assembly to retract the carrier further into the tubular housing.
19. The method according to clause 19, wherein the step of removing the cap comprises the following steps in the following order: rotating the cap relative to the tubular housing around the longitudinal axis; and pulling the cap in the proximal direction relative to the housing.

1-15 (canceled)

16. A sub-assembly of a medicament delivery device, the sub-assembly comprising:
a tubular housing;
a carrier;
a cap; and
a carrier retracting assembly,
wherein the tubular housing extends along a longitudinal axis between a distal end and a proximal end,
wherein the cap is removably attached to the tubular housing and at least partially enclosing the carrier,
wherein the carrier is configured to receive a medicament container of the medicament delivery device, and
wherein the carrier retracting assembly is configured to move the carrier relative to the tubular housing in the direction of the longitudinal axis from a proximal position where the carrier is at least partially arranged within the tubular housing to a distal position where the carrier is further into the tubular housing.

17. The sub-assembly according to claim 16, wherein the carrier retracting assembly comprises a pre-stressed resilient member, wherein the pre-stressed resilient member is arranged between the carrier and the tubular housing and is configured to extend in the direction of the longitudinal axis, wherein the carrier comprises a distally directed stop surface, wherein the sub-assembly comprises a proximally directed stop surface adjacent to the distally directed stop surface of the carrier when the cap is attached to the tubular housing and is offset relative to the distally directed stop surface during the removal of the cap from the tubular housing.

18. The sub-assembly according to claim 16, wherein the tubular housing comprises a fastener, wherein the cap comprises a counter fastener releasably attached to the fastener, and wherein the cap is movable relative to the tubular housing between an attached position where the fastener is fixed to the counter fastener and a detached position where the fastener is not fixed to the counter fastener.

19. The sub-assembly according to claim 18, wherein the fastener and the counter fastener form a bayonet engagement or a screw engagement.

20. The sub-assembly according to claim 17, wherein the proximally directed stop surface is arranged on the cap.

21. The sub-assembly according to claim 17, wherein the sub-assembly comprises an adapter attached to a part of the cap, wherein the proximally directed stop surface is arranged on the adapter, and wherein the adapter is rotatable relative to the carrier around the longitudinal axis.

22. The sub-assembly according to claim 17, wherein the proximally directed stop surface is arranged on the tubular housing, wherein the cap is rotationally fixed to the carrier by a rotational engagement, and wherein the cap is rotatable around the longitudinal axis relative to the tubular housing during the removal of the cap from the tubular housing.

23. The sub-assembly according to claim 16, wherein the carrier is rotationally fixed to the tubular housing by a rotational engagement.

24. The sub-assembly according to claim 22, wherein the rotational engagement is formed by at least one of a rib-and-rib engagement, a rib-and-recess engagement, and a rib-and-cut-out engagement.

25. The sub-assembly according to claim 17, wherein the tubular housing comprises a distally directed surface, wherein the carrier comprises a proximally directed surface, and wherein the pre-stressed resilient member engages with the distally directed surface of the tubular housing at a proximal end of the pre-stressed member and engages with the proximally directed surface of the carrier at a distal end of the pre-stressed member.

26. The sub-assembly according to claim 17, wherein the pre-stressed resilient member is a compression spring.

27. The sub-assembly according to claim 16, wherein the sub-assembly comprises a rod arranged within the tubular housing, wherein the rod extends along the longitudinal axis between a proximal end and a distal end, wherein the rod is fixed relative to the tubular housing in the direction of the longitudinal axis during the removal of the cap from the tubular housing, and wherein the proximal end of the rod is configured to move a stopper of the medicament container when the carrier is in the distal position.

28. The sub-assembly according to claim 27, wherein the rod is configured to be at least partially surrounded by a spring that is configured to expel the medicament contained within the medicament container.

29. A method of operating a medicament delivery device, the method comprising the steps of:
providing a medicament delivery device comprising a tubular housing, a carrier receiving a medicament container, a cap, and a carrier retracting assembly, wherein the tubular housing extends along a longitudinal axis between a distal end and a proximal end, and wherein the cap is removably attached to the tubular housing and at least partially encloses the carrier; and
removing the cap from the tubular housing, thereby triggering the carrier retracting assembly to retract the carrier further into the tubular housing.

30. The method according to claim 29, wherein the step of removing the cap comprises the following steps in the following order:
rotating the cap relative to the tubular housing around the longitudinal axis; and
pulling the cap in the proximal direction relative to the housing.
